(19) United States
(12) Patent Application Publication
(10) Pub. No.: US 2004/0225224 A1
(43) Pub. Date: Nov. 11, 2004

(54) Integrated inflating and quick releasing device for electronic sphygmomanometer

(76) Inventor: Daniel C.M. Tseng, Taipei Hsien (TW)

Correspondence Address: BIRCH STEWART KOLASCH & BIRCH, PO BOX 747, FALLS CHURCH, VA, 22040-0747, US

(21) Appl. No.: 10/428,942

(22) Filed: May 5, 2003

(51) Int. Cl. A61B 5/02

(52) U.S. Cl. 600/499

(57) ABSTRACT

An electronic sphygmomanometer, comprising a processor, a pressure sensor coupled to said processor, a cuff coupled to said pressure sensor, an inflating and quick releasing unit coupled to said cuff and a slow deflating device. The inflating and quick releasing unit has a driving element (motor), an inflating device, and a quick releasing device, and the driving element (motor) drives the inflating device in one direction to inflate air into the cuff and increase the pressure after receiving an inflating signal from the processor. The slow deflating device deflates the air in the cuff to adjust the pressure after the pressure reaches a predetermined setting. After the measurements are completed, the driving element (motor) instructs the quick releasing device in the inflating and quick releasing unit to rotate in the opposite direction to quickly discharge the air remaining in the cuff. Such arrangement constitutes an inflating and quick releasing device that can be inflated and quickly deflated.

### FIELD OF THE INVENTION

The present invention relates to an integrated inflating and quick releasing device for an electronic sphygmomanometer, more particularly to an integrated inflating and quick releasing device for electronic sphygmomanometer applicable to health and medical assessment.

### BACKGROUND OF THE INVENTION

Blood pressure is the force of blood against the walls of arteries. In general, the following physiological information can be obtained after blood pressure is measured:

For systolic pressure (as the heart beats) and diastolic pressure (as the heart relaxes between beats), the measurement is written one above or before the other, with the systolic number on top and the diastolic number on the bottom. For example, a blood pressure measurement of 120/80 mm Hg (millimeters of mercury) is expressed verbally as “120 over 80.”

(1) Systolic pressure: It is also called heart contraction pressure, which is the pressure measured while the heart is pumping blood into the blood vessel.

(2) Diastolic pressure: It is also called heart expansion pressure, which is the pressure measured while the heart is not contracting.

(3) Heartbeat: It is the number of heart contractions per minute.

Most of the electronic sphygmomanometers have an inflating device, a slow deflating device, and a quick releasing device. A processor is used to control the inflating device to inject air into a cuff. The pressure inside the cuff gradually increases and stops inflating when the pressure reaches a predetermined setting. The slow deflating device will then gradually release the air from the cuff to adjust the pressure. During this process, the pressure sensor will detect the pressure changes and output the results to the processor. The processor then analyzes the readings, calculates the values of systolic and diastolic pressures, and outputs the results to a displaying device. When the measurements of systolic pressure and diastolic pressure are completed, the quick releasing device is activated instantly to discharge the remaining air in the cuff immediately. However, most of the quick releasing devices are independent solenoid valves, and the size required by such solenoid valve is a limitation for reducing the size of the sphygmomanometer. Further, the quick releasing device also has an independent power supply for its operation, and thus consuming more energy.

### SUMMARY OF THE INVENTION

The primary objective of the present invention is to solve the aforementioned problems and eliminate the drawbacks of cited prior art by integrating the quick releasing device with the inflating device in order to reduce the size of the sphygmomanometer as well as reduce power consumption.

### BRIEF DESCRIPTION OF THE DRAWINGS

FIG. 1 is an illustrative diagram of the control flow chart of the present invention.

FIG. 2 is a perspective diagram of the disassembled parts of the structure of the present invention.

FIG. 3 is a cross-sectional diagram of the present invention.

FIGS. 4A and 4B are illustrative diagrams of the air holding and deflating movements of the present invention.

FIGS. 5A and 5B are illustrative diagrams of another preferred embodiment of the present invention.

FIGS. 6A and 6B are illustrative diagrams of a further preferred embodiment of the present invention.

### DETAILED DESCRIPTION OF THE PREFERRED EMBODIMENTS

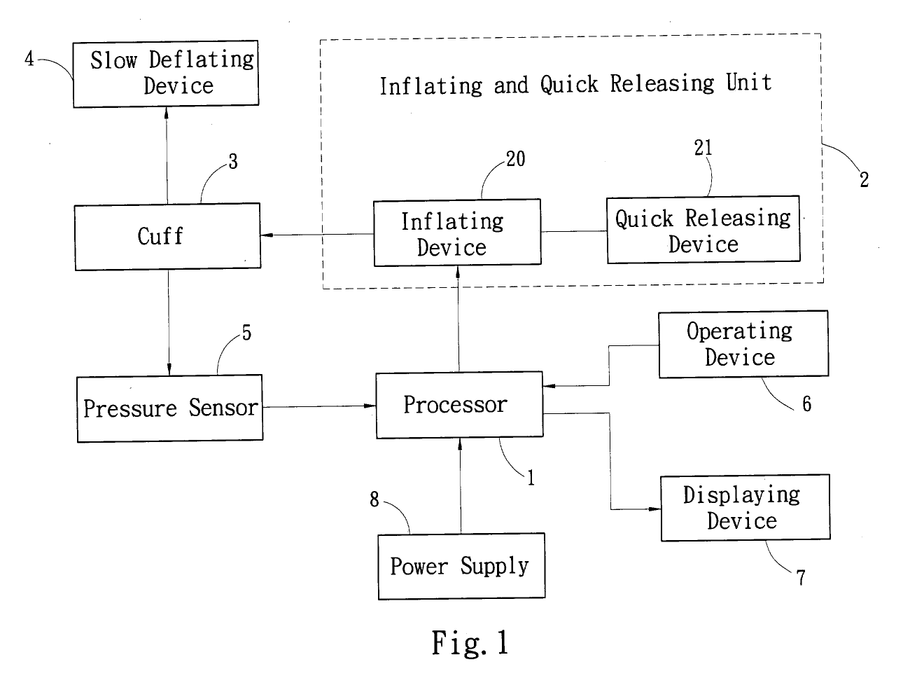

Please refer to FIG. 1 for the illustrative diagram of the control flow of the present invention. In the figure, it shows an inflating device, a slow deflating device, and a quick releasing device disposed in an electronic sphygmomanometer. In addition, the electronic sphygmomanometer of the present invention comprises a processor 1, a pressure sensor 5 coupled to said processor 1, a cuff 3 coupled to said pressure sensor 5 and the slow deflating device 4, and an inflating and quick releasing unit 2 within which are the inflating device 20 and the quick releasing device 21. The processor is also coupled to an external operating device 6, a displaying device 7, and an external power supply 8.

The external operating device 6 controls the processor 1 and the processor 1 outputs a signal to start the inflating device 20 in the inflating and quick releasing unit 2 to inflate the cuff device 3 until the pressure reaches a predetermined setting. Then, the pressure of the cuff 3 is gradually reduced as the slow deflating device 4 discharges the air and the pressure sensor 5 detects the pressure change in the cuff and sends the detected signal to the processor 1. Then the processor 1 performs a signal computation. If the calculations of the systolic pressure, diastolic pressure, and heartbeats are completed, the processor 1 will activate the quick releasing device 21 to discharge the remaining air in the cuff 3 quickly and send the measured blood pressure readings to the displaying device 7.

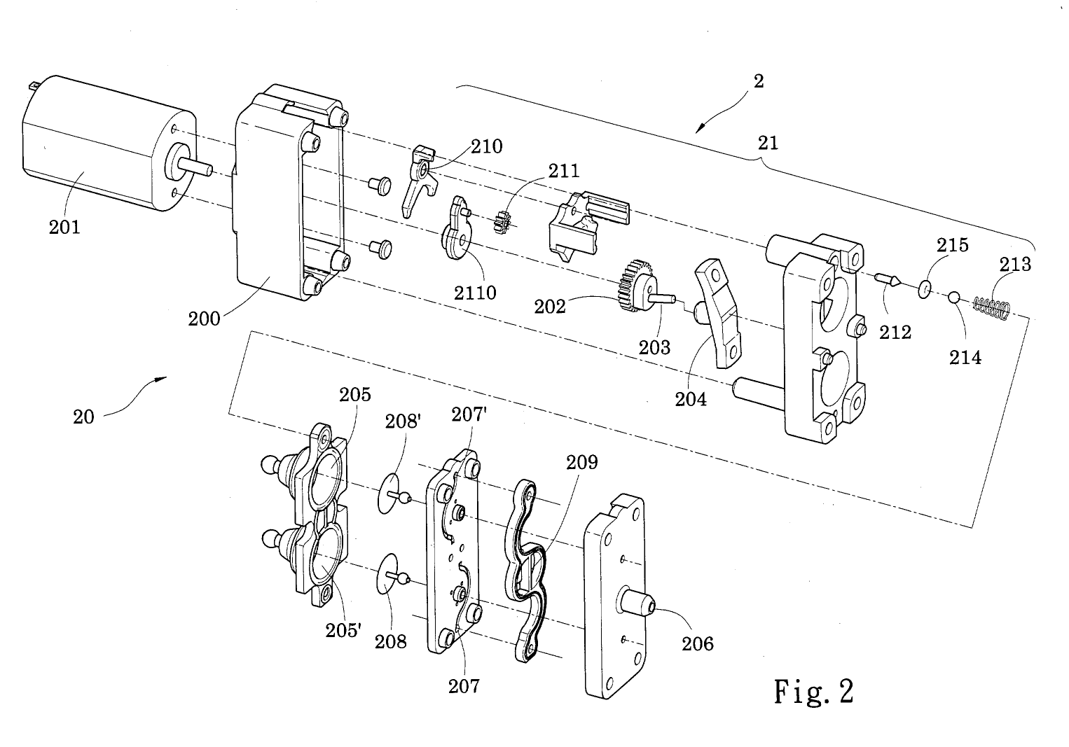

Please refer to FIG. 2 for the illustrative diagram of the disassembled parts of the structure of the present invention. The inflating and quick releasing unit 2 comprises an inflating device 20 and a quick releasing device 21. The inflating device 20 comprises a driving element 201 disposed in a housing 200, an active gear 202 disposed in said driving element 201, a motion rod 203 disposed on said active gear 202, a transmission member 204 coupled to said motion rod 203, air chambers 205, 205′ and an air outlet 206. An isolating member 209 is disposed between said air chambers 205, 205′ and air outlet 206; air holes 207, 207′ are connected to one side of the isolating member 209; the bottom of the air hole 207 is coupled to the external slow deflating device (not shown in the figure), wherein said driving element 201 is a motor, and said motion rod 203 is coupled to the transmission member 204 at a predetermined angle. Said quick releasing device 21 comprises a transmission gear 211 engaged with the active gear 202, a link rod 2110 coupled to said transmission gear 211, and a wedge member 210 adjacent to said link rod 2110, a prop member 212 adjacent to said wedge member 210, a sealing member 214 adjacent to said prop member 212, and an elastic member 213 disposed on another side of said sealing member 214, wherein said elastic member 213 is a spring, and said sealing member 214 is a sphere or other member having the same function of maintaining the airtight effect, and a spacer 215 is disposed between said prop member 212 and said sealing member 214.

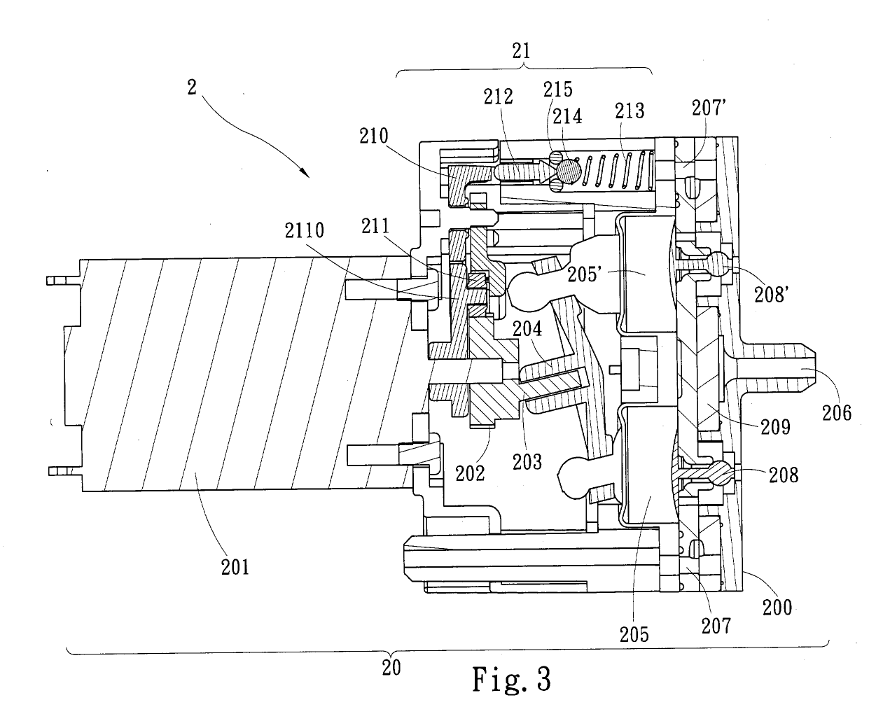

Please refer to FIG. 3 for the illustrative cross-sectional diagram of the present invention. In the figure, when said inflating and quick releasing unit 2 inflates air by the inflating device, the driving element 201 (motor) drives the main gear 202 to rotate in one direction (clockwise or counter-clockwise). Since the motion rod 203 is disposed on the main gear 202 at a predetermined angle, the transmission member 204 of the motion rod 203 also rotates at the predetermined angle, and the transmission member 204 will drive the air chambers 205, 205′ to contract and expand for the movement such that the inlet valves 208, 208′ produce the incoming air and seal the air, and the compressed air will push the air out from the air outlet 206 to the cuff 3. The quick releasing device remains in an airtight position during the above process. For the quick release of the remaining air in the cuff after measurement is completed, the driving element 201 drives the main gear 202 to rotate in the opposite direction and said transmission mechanism is in the opposite direction, therefore such wedge member 210 will be kept at the highest point and push the prop member 212. Such prop member 212 will push the sealing member 214 upward and detach from the spacer 215. The sealing member 214 presses against the elastic member 213 so that the quick releasing device 21 will have an opening, and air will pass through the air hole 207′ and leak from the side of the sealing member 214 to achieve the effect of quick deflation.

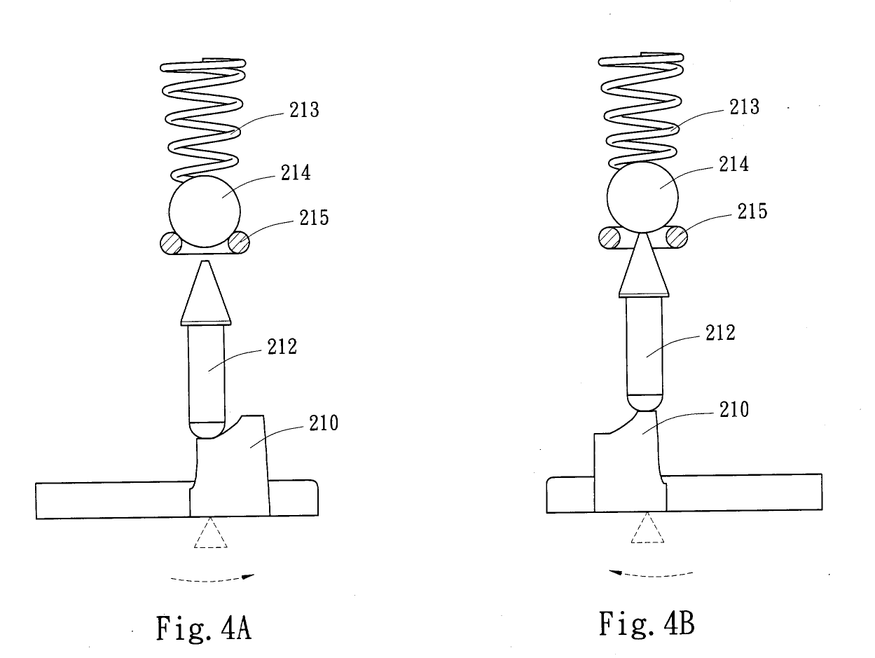

Please refer to FIGS. 4A and 4B for the illustrative diagrams of the inflating and deflating movements. In the figure, the top of the wedge member 210 has an aslant surface to divide the top surface of the wedge member 210 into the lowest point and the highest point. When the prop member 212 is disposed at the lowest point of the wedge member 210, the prop member 212 cannot press the sealing member 214 and the elastic member 213 will in turn press the prop member 212 and the spacer 215 such that the upper chamber is kept sealed. When the wedge member 210 rotates to keep the prop member 212 at the highest point of the wedge member 210 and the prop member 212 pushes the sealing member 214 up and detach from the spacer 215, the quick releasing device 21 has an opening. The air in the cuff will pass through the air hole 207′ and be discharged from the sealing member 214 to achieve the effect of quick deflation.

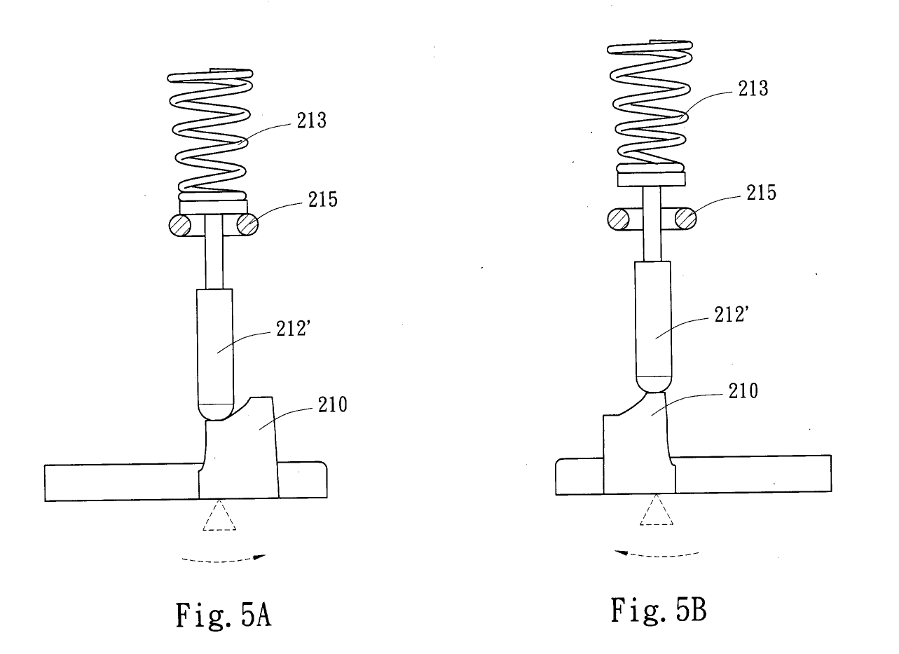

Please refer to FIGS. 5A and 5B for the illustrative diagrams of another preferred embodiment. In the figure, the prop member 212 and the sealing member 214 in said quick releasing device 21 can be combined into a prop member 212′ to achieve the same function.

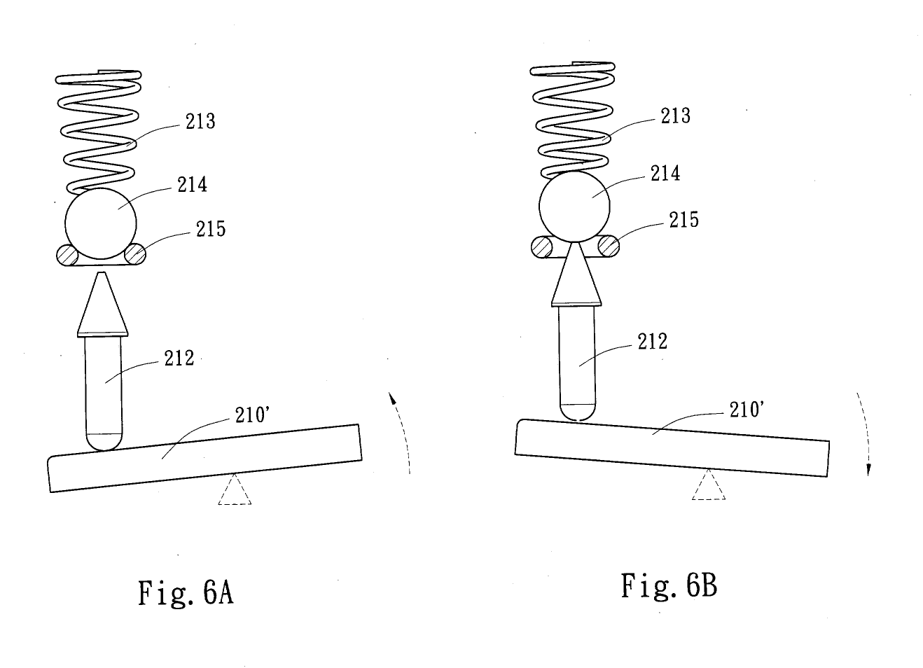

Please refer to FIGS. 6A and 6B for the illustrative diagrams of a further preferred embodiment. In the figure, the operation of the wedge 210 in said quick releasing device 21 can use the rising and dropping movement of the lever device 210′ to achieve the purpose of deflating quickly or air sealing.

What is claimed is:

1. An integrated inflating and quick releasing unit for electronic sphygmomanometer being coupled to a processor, a cuff, a slow deflating device and a pressure sensor, characterized in that:
said inflating device receives a signal from the processor, the inflating device is driven in one direction to inflate the cuff and increase the pressure until the pressure reaches a predetermined setting; the pressure of the cuff is then reduced gradually as the slow deflating device discharges the air; the pressure sensor transmits the signal to the processor to start blood pressure measurement; after the systolic pressure, diastolic pressure, and heartbeats are measured and calculated, the processor instructs the inflating device to rotate in the opposite direction for the quick releasing device in the inflating and quick releasing unit to quickly discharge the remaining air in the cuff.

2. The integrated inflating and quick releasing unit for electronic sphygmomanometer of claim 1, wherein said inflating device comprises a driving unit, a main gear disposed on said driving unit, a motion rod disposed on said main gear, a transmission member coupled to said motion rod, an air inlet valve and an air outlet coupled to said transmission member.

3. The integrated inflating and quick releasing unit for electronic sphygmomanometer of claim 1, wherein said quick releasing device comprises a transmission gear engaged to a main gear, a link rod coupled to said transmission gear, a wedge member disposed adjacent to said link rod, a prop member disposed adjacently to said wedge member, a sealing member disposed adjacently to said prop member, and an elastic member disposed on another side of the sealing element.

4. The integrated inflating and quick releasing unit for electronic sphygmomanometer of claim 3, wherein said elastic member is a spring.

5. The integrated inflating and quick releasing unit for electronic sphygmomanometer of claim 3, wherein said sealing member is one selected from the collection of a spherical member and a cylindrical member integrated with said prop member and said sealing member as a whole.

6. The integrated inflating and quick releasing unit for electronic sphygmomanometer of claim 3, wherein said wedge member is a lever device.

7. The integrated inflating and quick releasing unit for electronic sphygmomanometer of claim 1, wherein said unit drives the inflating device in one direction to inflate the cuff and increase the pressure, and in the opposite direction to discharge the pressure in the cuff by the quick releasing device.
